(19) United States
(12) Patent Application Publication
(10) Pub. No.: US 2026/0090912 A1
(43) Pub. Date: Apr. 2, 2026

(54) LOW POWER ELECTRONIC VISION SYSTEM USING OCULAR DEVICE

(71) Applicant: GLAIVE MEDICAL OPTICS, INC., Carlisle, MA (US)

(72) Inventors: Wayne H. WOODS, JR., Carlisle, MA (US); Alexander J. MELVILLE, Chelmsford, MA (US); Christopher SHELBY, Shreveport, LA (US)

(21) Appl. No.: 19/341,757

(22) Filed: Sep. 26, 2025

Related U.S. Application Data
(60) Provisional application No. 63/699,256, filed on Sep. 26, 2024.
(60) Provisional application No. 63/699,293, filed on Sep. 26, 2024.
(60) Provisional application No. 63/699,295, filed on Sep. 26, 2024.

(51) Int. Cl. A61F 9/00; A61F 9/08

(52) U.S. Cl. CPC A61F 9/0017; A61F 9/08

(57) ABSTRACT

An electronic ocular device is configured to be implanted in an eye and includes: multiple imaging systems configured to receive incoming light at the eye; and multiple projection systems configured to simultaneously project multiple different images onto multiple different regions of a retina of the eye based on the incoming light received by the imaging systems. Power used by the electronic ocular device is minimized by operating different ones of the projection systems at different resolutions based on different eye resolution at different regions of the retina.

### RELATED APPLICATIONS

The application claims priority to U.S. provisional patent application No. 63/699,256, filed Sep. 26, 2024, U.S. provisional patent application No. 63/699,293, filed Sep. 26, 2024, and U.S. provisional patent application No. 63/699,295, filed Sep. 26, 2024, the contents of each of which are incorporated by reference herein in their entirety.

### BACKGROUND

The present disclosure relates to electronic vision systems. Such systems have a variety of application such as therapeutic (e.g., to treat vision disorders such as blindness), professional, and recreation.

### SUMMARY

In accordance with aspects of the present disclosure, a low power electronic vision system includes an ocular device that is configured to simultaneously project frontal vision images onto a first region of a retina at a relatively high resolution and peripheral vision images onto a second region of the retina at relatively low resolution. In various implementations, the ocular device includes a first imaging system and a first projection system that are configured to receive incoming light associated with frontal vision and project images corresponding to this incoming light at the frontal vision region of the retina. In various implementations, the ocular device additionally includes a second imaging system and a second projection system that are configured to receive incoming light associated with peripheral vision and project images corresponding to this incoming light at the peripheral vision region of the retina. Because the eye detects frontal vision and peripheral vision at different levels of detail, and because the frontal vision region of the retina is a different size than the peripheral vision region of the retina, embodiments of the present disclosure are optimized to minimize the number of pixels used in the respective projection systems when simultaneously projecting frontal vision and peripheral vision images onto the retina. Minimizing the number of pixels used, while simultaneously providing adequate frontal and peripheral vision, enables reducing the physical size of the ocular implant and reducing the electrical power consumed by the ocular implant, both of which are desirable. In this manner, blindness from a multitude of disparate sources can be treated, including eye injuries, retinal diseases, and age-related degeneration.

In an aspect of the present disclosure, there is an electronic device configured to be implanted in or on an eye, the electronic device comprising: multiple imaging systems configured to receive incoming light at the eye; and multiple projection systems configured to simultaneously project multiple different images onto multiple different regions of a retina of the eye based on the incoming light received by the imaging systems.

In various embodiments of the electronic device, the different regions of the retina include: a first region associated with frontal vision; and a second region associated with peripheral vision.

In various embodiments of the electronic device, the projection systems comprise: a first projection system configured to project first images onto the first region at a first resolution; and a second projection system configured to project second images onto the second region at a second resolution that is lower than the first resolution.

In various embodiments of the electronic device, respective ones of the different regions are defined based on respective ranges of off-axis angles in the eye.

In various embodiments of the electronic device, the imaging systems share a single imaging sensor and each respective one of the imaging systems has a respective imaging lens over a respective portion of the single imaging sensor.

In various embodiments of the electronic device, the projection systems share a single light projection device and each respective one of the projection systems has a respective projection lens over a respective portion of the single light generation device.

In various embodiments of the electronic device, each respective one of the imaging systems has a respective imaging lens and a respective imaging sensor.

In various embodiments of the electronic device, different ones of the respective imaging sensors have different numbers of pixels.

In various embodiments of the electronic device, each respective one of the projection systems has a respective projection lens and a respective light generation device.

In various embodiments of the electronic device, different ones of the respective light generation devices have different numbers of pixels.

In various embodiments of the electronic device, the electronic device is configured to be implanted in a capsular bag of the eye, a ciliary sulcus of the eye, or an anterior chamber of the eye.

In various embodiments of the electronic device, the electronic device comprises a central body and haptics extending from the central body.

In various embodiments of the electronic device, the imaging systems and the projection systems are contained in a chip or chip stack, or attached to a printed circuit board, in the central body.

In various embodiments of the electronic device, the electronic device further comprises control circuitry, a power source, a wireless communication system, and a wireless charging system.

In various embodiments of the electronic device, at least two of the imaging systems are configured to receive light coming from outside the eye, and at least one of the imaging systems is configured to receive light coming from inside the eye.

In accordance with aspects of the present disclosure, a method comprises determining a damaged area of the retina by analyzing images defined by the light received by the at least one of the imaging systems in various embodiments of the electronic device.

In accordance with aspects of the present disclosure, a method of using various embodiments of the electronic device comprises: projecting the first images using a first a light generation device in the first projection system; and projecting the second images using a second a light generation device in the second projection system, wherein the projecting the second images comprises not using pixels in the second light generation device that correspond to the first region. The projecting the second images may comprise not using other pixels in the second light generation device that correspond to a damaged region of the retina.

In various embodiments of the electronic device, the multiple different regions of the retina include: a first region associated with frontal vision; a second region associated with peripheral vision; and a middle region between the first region and the second region.

In various embodiments of the electronic device, the multiple projection systems comprise: a first projection system configured to project images onto the first region at a first resolution; a second projection system configured to project images onto the second region at a second resolution that is less than the first resolution; and a third projection system configured to project images onto the middle region at a third resolution that is less than the first resolution and greater than the second resolution.

In various embodiments of the electronic device, the electronic device comprises one of: a contact lens that is configured to be placed on an exterior of the eye; or an implantable contact lens that is configured to be implanted in the eye.

### BRIEF DESCRIPTION OF THE DRAWINGS

Various features and advantages of the technology of the present disclosure will be apparent from the following description of particular examples of those technologies, and as illustrated in the accompanying drawings. The drawings are not necessarily to scale; the emphasis instead is placed on illustrating the principles of the technological concepts. In the drawings, like reference characters may refer to the same parts throughout the different views. The drawings depict only illustrative examples of the present disclosure and are not limiting in scope.

FIG. 1 shows a diagram of an eye with an implanted ocular device in accordance with aspects of the present disclosure.

FIG. 2 diagrammatically shows a first region and a second region on a portion of a retina in accordance with aspects of the present disclosure.

FIG. 3 shows an exemplary implementation of the ocular device of FIG. 1 in accordance with aspects of the present disclosure.

FIG. 4 shows diagrammatic cross-sectional views of components of the ocular device of FIG. 1 in accordance with aspects of the present disclosure.

FIG. 5 shows an example of an ocular device implanted in an eye in accordance with aspects of the present disclosure.

FIG. 6 diagrammatically shows an exemplary implementation of an ocular device in accordance with aspects of the present disclosure.

FIG. 7 diagrammatically shows an example of projection regions on the retina using the ocular device of FIG. 6 in accordance with aspects of the present disclosure.

FIG. 8 diagrammatically shows another example of projection regions on the retina using the ocular device of FIG. 6 in accordance with aspects of the present disclosure.

FIG. 9 diagrammatically shows an exemplary arrangement of the imaging systems of ocular device of FIGS. 6 and 8 in accordance with aspects of the present disclosure.

FIG. 10 diagrammatically shows an exemplary arrangement of imaging systems from FIG. 9 in accordance with aspects of the present disclosure.

FIG. 11 diagrammatically shows an example of an ocular device accommodating a damaged area of the retina in accordance with aspects of the present disclosure.

FIG. 12 shows a flowchart of an exemplary method in accordance with aspects of the present disclosure.

FIG. 13 shows a flowchart of an exemplary method in accordance with aspects of the present disclosure.

FIG. 14 shows a plot of eye resolution versus off-axis angle in accordance with aspects of the present disclosure.

FIG. 15 illustrates wireless charging in accordance with aspects of the present disclosure.

### DETAILED DESCRIPTION

The particulars shown herein are by way of example and for purposes of illustrative discussion of the embodiments of the present disclosure only and are presented in the cause of providing what is believed to be the most useful and readily understood description of the principles and conceptual aspects of the present disclosure. In this regard, no attempt is made to show structural details of the present disclosure in more detail than is necessary for the fundamental understanding of the present disclosure, the description taken with the drawings making apparent to those skilled in the art how the several forms of the present disclosure may be embodied in practice.

Various embodiment of the present disclosure include an implantable electronic ocular device that utilizes multiple imaging systems with multiple projection systems to simultaneously project frontal vision images and peripheral vison images onto a retina while to minimizing power and pixels used to perform such image projections. Implementations achieve this minimizing by making use of natural resolution versus angle profile of the human retina. The human retina has a very high resolution (“20/20” vision) of about 60 pixels/degree, but this high resolution is only in a very narrow angular range on the retina: roughly +/−1.5 degrees from the center of the retina. However, the vast majority of the retinal tissue has a much lower resolution. For example, the areas of the retina nearer to 50 to 60 degrees off center is down around 2 pixels per degree. This makes intuitive sense as we know that the peripheral vision has far less resolution than the frontal vision. For a projection system concerned with augmented reality, this is not an issue: such systems focus their projections primarily to overlay data over the frontal vision of the patient. However, for applications that are trying to provide or augment the peripheral vision of the patient, peripheral projection is of particular importance. Given that it is important to project to the frontal vision and the peripheral vision, one does not have great choices in how to do so. For example, if a single lens/screen projection system is used, then one must project at 60 pixels/degree to allow the maximum forward vision resolution. To project that resolution across +/−60 degrees would require 120 degrees×60 pixels/degree or 7200 pixels which would require 7200 pixels in X and Y which is 7200×7200 pixels or over 50 million pixels. If, alternatively, one used a single lens/screen system to only project at the low-resolution level of the far-peripheral area of the retina, then this would require 2 pixels/degree×120 degrees or 240 pixels, leading to 240×240 pixels or 57,600 pixels. However, if projecting only in low resolution, then the forward vision no longer has any high-resolution vision. Both of these solutions are undesirable because in the first scenario there is way more power/pixels being used that cannot even be resolved in the peripheral areas, and in the second case there is no longer any high-resolution imaging possible.

Implementations according to the present disclosure provide a solution to this problem by using multiple projection systems to simultaneously project images to different areas of the eye. The relatively small area of the retina that is associated with high-resolution frontal vision is projected to with a high-resolution projection system, while the relatively large area of the retina that is associated with low-resolution peripheral vision is projected to with a low-resolution projection system. In this manner, the overall number of pixels included in, and powered by, the ocular implant can be greatly reduced while simultaneously providing perfectly adequate frontal and peripheral vision to the user.

FIG. 14 shows a plot 1400 of eye resolution versus off-axis angle in accordance with aspects of the present disclosure. In FIG. 14, the curved line represents resolution of the eye plotted against the off-axis angle, which is the angle from the pupil between the optical axis of the eye and the retina. Embodiments of the present disclosure include an ocular device having different projection systems with different lenses designed to minimize the number of pixels while maximizing the patient's restored vision, represented in FIG. 14 as Lenses A, B, and C, providing peripheral vision, mid-field of view vision, and central vision, respectively

An ocular device in accordance with aspects of the present disclosure and having different projection systems with lenses A, B, and C as shown in FIG. 14 provides using far less pixels than 50 million pixels as would be the case with a single resolution system. For example, in an ocular device according to FIG. 14, a first projection system having lens C projects relatively high resolution (20/40 vision) or 30 pixels/degree at +/−2 degrees off-axis, which is a total of 4 degrees×30 pixels/degree leading to a need of 120×120=14,400 pixels for this first region. In this example of an ocular device according to FIG. 14, a second projection system having lens B projects a middle resolution of 12 pixels per degree over +/−15 degrees leading to 360×360 pixels or 129,600 pixels in this second region. In this example of an ocular device according to FIG. 14, a third projection system having lens A projects a lowest resolution of 5 pixels per degree over +/−60 degrees leading to 720×720 pixels or 518,500 pixels in this third region. This example of an ocular device according to FIG. 14, having three different systems projection images at three different resolutions, covers the entire retinal area with an approximation of the eye's natural resolution profile. This three system approach (note this could be done with a single screen and three lenses as well), requires only 662,400 pixels as opposed to 51,840,000 pixels for a single lens that can cover the whole retinal area and allow high-resolution forward vision. This means that for about 1% of the pixels and power, the eye can be provided with a good approximation of a visual field. Moreover, as described herein, this pixel count (i.e., 662,400 pixels) can be further reduced because the middle pixels of the middle and low resolution systems (i.e., pixels correspond to areas where projection regions overlap) are not needed and can be selectively turned off to avoid image overlap and further reduce power consumption of the ocular device. Various embodiments utilize this same reasoning on the imaging (e.g., camera) side of the ocular device and, thus, may utilize imaging systems having different resolutions to reduce pixel count on the image sensors, since there is there is little point in taking in more data that the eye can resolve. In this manner, implementations having multiple imaging systems and multiple projection systems, each tailored to different resolutions based on differing eye resolutions, drastically reduces pixel count and power required in the ocular device.

In the technical field of electronic ocular implants, embodiments of the present disclosure provide the advantage minimized power because using less pixels results in less power consumed by the projection systems (e.g., LED power), using less pixels results in less power consumed by the imaging systems (e.g., CMOS imager power), and using less pixels results in less internal data transfer (about 1% of what would otherwise be needed) between the imaging and projection systems which means much less bandwidth and data transfer power required.

Some embodiments use multiple different imaging systems each having a respective image sensor (e.g., CMOS image sensor) and lens, and multiple different projection systems each having a respective light projecting device (e.g., LED panel) and lens. Other embodiments used a single shared image sensor for the multiple imaging systems (e.g., multiple different lenses arranged over different sections of a single CMOS image sensor) and a shared light projecting device for the multiple projection systems (e.g., multiple different lenses arranged over different sections of a single LED panel). In the latter, power savings can be achieved by selectively turning off varying amounts of pixels in the different sections of the LED panel based on the different resolutions of projection associated with the different sections of the LED panel. These embodiment also benefit from case of manufacturing since only a single CMOS image sensor and a single LED panel are used.

Implementations in accordance with aspects of the present disclosure thus may be used to provide an electronic ocular implant including a multiple lens/periscope/prism system on the projection side and a multiple lens/periscope/prism system on image side. The electronic ocular implant may be used to perform a method of optimally projection images onto the frontal and peripheral regiones of the retina. As described herein, the electronic ocular implant may also be used to perform a method of optimally imaging the retina of the eye in which the ocular device is implanted.

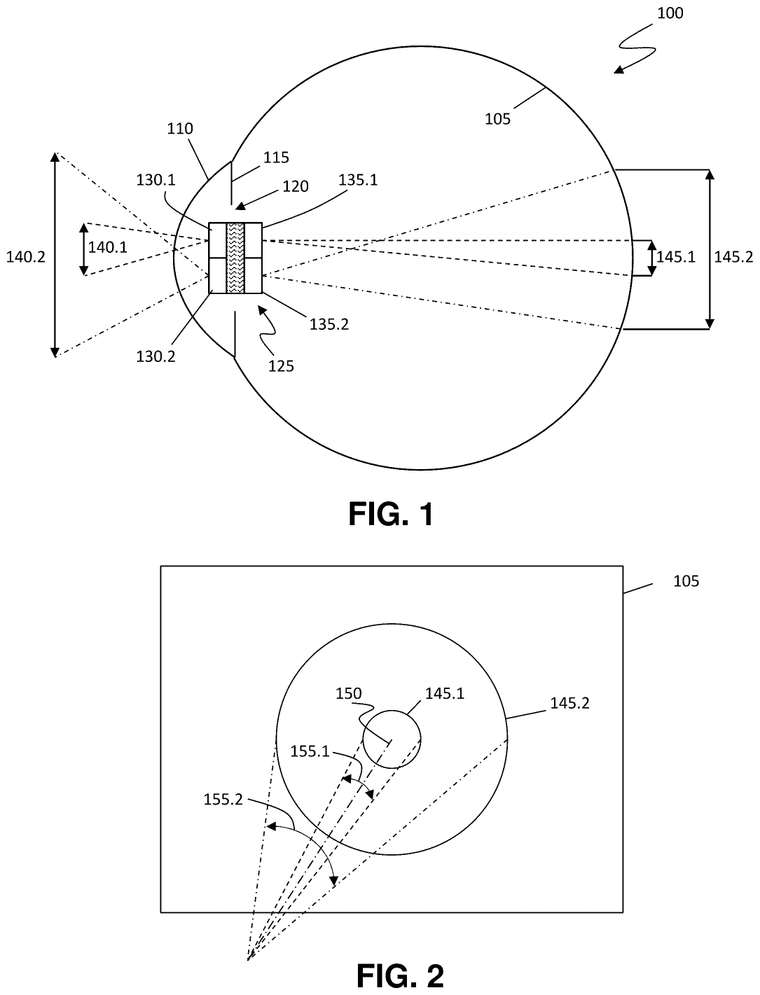

FIG. 1 shows a diagram of an eye 100 with an implanted ocular device 125 in accordance with aspects of the present disclosure. The eye 100 includes a retina 105 at a posterior region and a cornea 110, iris 115, and pupil 120 at an anterior region.

An ocular device 125 in accordance with aspects of the present disclosure is implanted in the eye 100 at the anterior region, e.g., at the capsular bag of the eye 100, the ciliary sulcus of an eye 100, or the anterior chamber of an eye 100. The ocular device 125 comprises multiple imaging systems and multiple projection systems. In the example shown in FIG. 1, the ocular device 125 includes two electronic imaging systems (i.e., a first imaging system 130.1 and a second imaging system 130.2) and two electronic projection systems (i.e., a first projection system 135.1 and a second projection system 135.2). An ocular device in accordance with aspects of the present disclosure is not limited to two imaging systems and two projection systems, and other numbers of each type of system may be used in different implementations.

In accordance with aspects of the present disclosure, the first imaging system 130.1 is associated with and operatively connected to the first projection system 135.1, the second imaging system 130.2 is associated with and operatively connected to the second projection system 135.2, and the ocular device 125 is arranged such that: the first imaging system 130.1 is configured to receive incoming light from a first field of view 140.1 outside the eye 100 and the first projection system 135.1 is configured to project light onto a first region 145.1 of the retina 105 at a first resolution based on signals generated by the first imaging system 130.1 in response to light received by the first imaging system 130.1; and the second imaging system 130.2 is configured to receive incoming light from a second field of view 140.2 outside the eye 100 and the second projection system 135.2 is configured to project light onto a second region 145.2 of the retina 105 at a second resolution (that is different than the first resolution) based on signals generated by the second imaging system 130.2 in response to light received by the second imaging system 130.2.

In accordance with aspects of the present disclosure, the first region 145.1 of the retina 105 is a region of the retina 105 that includes receptors for frontal vision, and the second region 145.2 of the retina 105 is a region of the retina that includes receptors for peripheral vision. In one example, the first region 145.1 of the retina 105 includes primarily cones and is in the fovea at the central region of the retina 105, and the second region 145.2 of the retina 105 includes primarily rods and is in the outer regions of the retina 105 surrounding the fovea. Cones are used for frontal vision and have high acuity which allows for sharp, detailed vision. Rods are used for peripheral vision and have low acuity that provides less sharp and less detailed vision than achieved with cones.

In accordance with aspects of the present disclosure, the first projection system 135.1 is configured to project light onto the first region 145.1 at a first resolution that is relatively high, and the second projection system 135.2 is configured to project light onto the second region 145.2 at a second resolution that is relatively low, where the first resolution is higher than the second resolution. In embodiments, the first resolution is relatively high in order to provide more detailed images for the user's frontal vision, and the second resolution is relatively low in order to provide less detailed images for the user's peripheral vision. In embodiments, the number of pixels used in the first projection system 135.1 is tailored to the size (e.g., area) of the first region 145.1 and the magnitude of the first resolution, and the number of pixels used in the second projection system 135.2 is tailored to the size (e.g., area) of the second region 145.2 and the magnitude of the second resolution. In this manner, each of the first projection system 135.1 and the second projection system 135.2 may be optimized to minimize its physical size and power consumption based on the respective numbers of pixels used by the respective projection systems.

FIG. 2 diagrammatically shows the first region 145.1 and the second region 145.2 on a portion of the retina 105, looking inward along an axis 150 that intersects the fovea of the eye 100. In some embodiments, the first region 145.1 and the second region 145.2 may be defined based on an off-axis angle, which as used herein refers to the angle defined between the axis 150 extending from the center of the pupil to the center of the fovea and a line extending from the center of the pupil to another point on the retina. In one example, the first region 145.1 is the area of the retina 105 defined within a first range of off-axis angles 155.1 (e.g., off-axis angles within a range of +/−2 degrees), the first resolution is 30 pixels per degree, the second region 145.2 is the area of the retina 105 defined within a second range of off-axis angle 155.2 (e.g., off-axis angles within a range of +/−60) degrees and not including the first region 145.1, and the second resolution is 5 pixels per degree. In this manner, the ocular device 125 is configured to project a relatively high-resolution image on the first region 145.1 defined by the first off-axis angle 155.1, and to project a relatively low-resolution image on the second region 145.2 defined by the second off-axis angle 155.2.

In various embodiments, the ocular device 125 stores data that is based on a mapping of the retina 105 for a particular eye of a particular user and that is used to control the operation of the first projection system 135.1 and the second projection system 135.2 so that the first projection system 135.1 projects light only onto the first region 145.1 and so that the second projection system 135.2 projects light only onto the second region 145.2. In embodiments, the second projection system 135.2 does not project light onto the first region 145.1 so as to avoid overlap of projected images in the first region 145.1. This may be accomplished, in one example, by turning off (e.g., not utilizing) light emitting pixels in the second projection system 135.2 that are mapped to the first region 145.1.

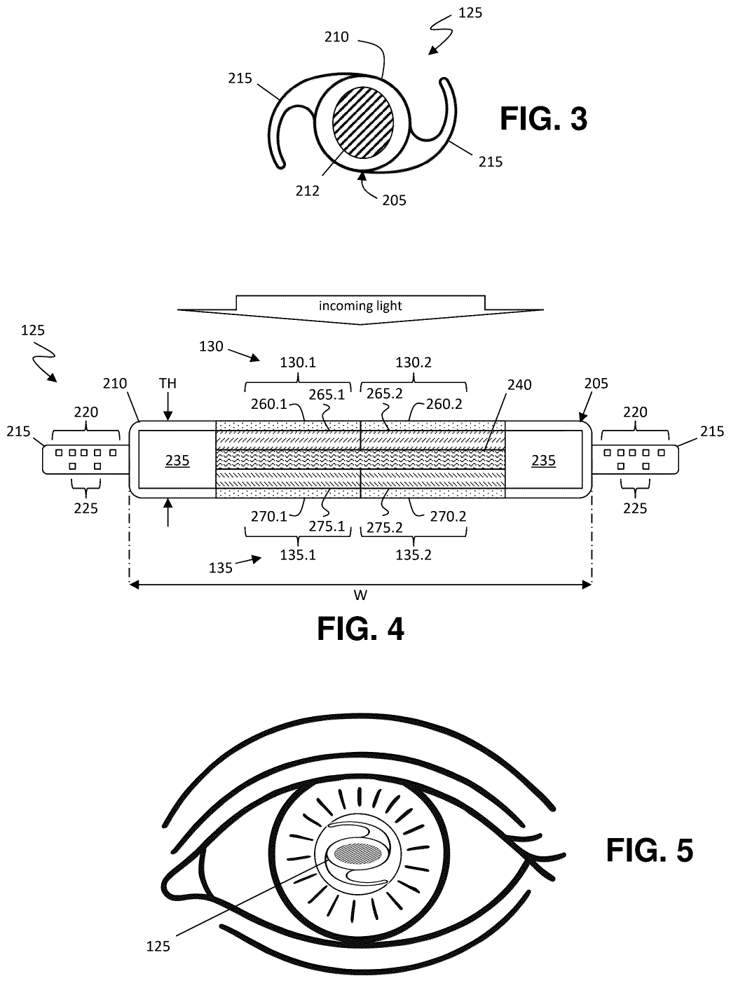

FIG. 3 shows an exemplary implementation of the ocular device 125 of FIG. 1. In embodiments, the ocular device 125 is in the form of an electronic device that is configured to be implanted in the eye of a user and comprises a body 205 that has a central portion 210 and haptics 215 with electronics 212 in the body 205. The ocular device 125 is depicted diagrammatically in FIG. 3 and is not limited to the shape shown in FIG. 3. For example, the central portion 210 and/or the haptics 215 of the ocular device 125 may be shaped differently than shown in FIG. 3.

FIG. 4 shows a diagrammatic cross-sectional view of the ocular device 125 of FIG. 3 in accordance with aspects of the present disclosure. In embodiments, and as shown in FIGS. 3 and 4, the body 205 comprises haptics 215 in the form of wings or tabs that each extend outward from the central portion 210. The body 205 may be made in the form of a single piece composed of materials such as acrylic and/or silicone lens material that encapsulates the electronics 212. In another example, the central body 205 may be made in the form of a multi-piece (e.g., two piece) case that can be opened and closed and that houses the electronics of the ocular device 125.

In embodiments, the electronics 212 include a wireless charging coil 220, a wireless communication antenna 225, multiple imaging systems 130 (e.g., first imaging system 130.1 and second imaging system 130.2), multiple projection systems 135 (e.g., first projection system 135.1 and second projection system 135.2), a power source 235, and control circuitry 240. In some configurations, the charging coil 220 and the wireless communication antenna 225 are embedded in one or both of the haptics 215 and the imaging systems, projection systems, power source 235, and control circuitry 240 are integrated in chip stack contained in the central portion 210 of the body 205, although other arrangements may be utilized. In some embodiments, the functions of the charging coil 220 and the wireless communication antenna 225 are combined in a single coil of electrical conductor (e.g., wire). In some embodiments, the charging coil 220 and the wireless communication antenna 225 are formed in the shape of a coil wrapped around an interior of the body 205.

In accordance with aspects of the present disclosure, respective ones of the imaging systems 130 are configured to receive incoming light from outside the eye when the ocular device 125 is implanted in an eye of a user, e.g., as shown in FIGS. 1 and 5. In embodiments, each respective one of the imaging systems comprises one or more image sensors that receive incoming light from outside the eye and that provide input to the control circuitry 240 based on the received light. The image sensors may comprise any suitable type of on-chip imaging technology, such as a charge-coupled device (CCD) or a complementary metal-oxide-semiconductor (CMOS) image sensor. The respective ones of the imaging systems may comprise different regions of a single image sensor or may comprise separate image sensors. Each respective one of the imaging systems may include one or more specialized local lens structures to enhance functionality of the ocular device 125. For example, the first imaging system 130.1 may comprise a first imaging lens 260.1 and a first image sensor 265.1, the first imaging lens 260.1 having first optical characteristics (e.g., a standard-angle lens). In this example, the second imaging system 130.2 may comprise a second imaging lens 260.2 and a second image sensor 265.2, the second imaging lens 260.2 having second optical characteristics (e.g., a wide-angle lens) different than the first optical characteristics. In some embodiments, the output of each respective imaging system is a respective time-dependent electronic signal to the control circuitry 240 that corresponds to incoming light received at the respective imaging system.

In accordance with aspects of the present disclosure, respective ones of the projection systems are configured to project light (e.g., images) onto respective defined regions of the retina, such as the first region 145.1 and second region 145.2 as described with respect to FIGS. 1 and 2, in one example. In embodiments, each respective one of the projection systems receives respective control signals from the control circuitry 240 and projects a respective image onto a respective region of the retina based on the control signals. The projection systems may comprise any suitable light generation devices, such as a light generation panel comprising light emitting diodes (LEDs), as but one example. The respective ones of the projection systems may comprise different regions of a single light generation device or may comprise separate light generation devices.

Each respective one of the projection systems 135 may include one or more specialized local lens structures to enhance functionality of the ocular device 125. For example, the first projection system 135.1 may comprise a first projection lens 270.1 and a first light generation device 275.1 (e.g., a first LED panel including a first number of pixels at a first pitch), the first projection lens 270.1 having first optical characteristics (e.g., a standard-angle lens). In this example, the second projection system 135.2 may comprise a second projection lens 270.2 and a second light generation device 275.2 (e.g., a second LED panel including a second number of pixels at a second pitch), the second projection lens 270.2 having second optical characteristics (e.g., a wide-angle lens) different than the first optical characteristics.

In embodiments, the projection lens used in a respective one of the projection systems 135 is optically tailored to the resolution displayed by the respective projection system and size of the region projected onto by the respective projection system. For example, a first projection lens included in the first projection system 135.1 may be manufactured with first optical characteristics (e.g., focal length, field of view, refractive index, etc.) that are specifically configured to deliver an image onto an area the size of the first region 145.1 at the first resolution used by the first projection system 135.1, and a second projection lens included in the second projection system 135.2 may be manufactured with second optical characteristics (e.g., focal length, field of view, refractive index, etc.) that are different from the first optical characteristics and that are specifically configured to deliver an image onto an area the size of the second region 145.2 at the second resolution used by the second projection system 135.2.

The ocular device 125 may be structured and arranged with one or more of the projection lenses (e.g., first projection lens 265.1 and second projection lens 265.2) to provide linear path projection as described in U.S. application Ser. No. 19/195,967, filed May 1, 2025, titled “LINEAR PATH IMAGE PROJECTION ONTO RETINA FROM ELECTRONIC INTRAOCULAR LENS (IOL)” and published as US patent application Publication No. [to be added after publication], the contents of which are incorporated by reference herein in their entirety. For example, one or more of the projection systems (e.g., the first projection system 135.1) may include an enclosure, lens (e.g., first projection lens 270.1), and display (e.g., first light generation device 275.1) arranged in a manner described in U.S. application Ser. No. 19/195,967.

Additionally or alternatively, the ocular device 125 may be structured and arranged with one or more of the projection lenses (e.g., first projection lens 265.1 and second projection lens 265.2) to provide circuitous path projection as described in U.S. application Ser. No. 19/195,971, filed May 1, 2025, titled “CIRCUITOUS PATH IMAGE PROJECTION ONTO RETINA FROM ELECTRONIC INTRAOCULAR LENS (IOL)” and published as US patent application Publication No. [to be added after publication], the contents of which are incorporated by reference herein in their entirety. For example, one or more of the projection systems (e.g., the first projection system 135.1) may include an enclosure, lens (e.g., first projection lens 270.1), and display (e.g., first light generation device 275.1) arranged in a manner described in U.S. application Ser. No. 19/195,971.

The control circuitry 240 may comprise a controller, a central processing unit (CPU), an application-specific integrated circuit (ASIC), a field-programmable gate array (FPGA), a system-on-chip (SoC), or other processing device or computing device for processing executable instructions. The electronics 212 may be composed of sub-circuits which may be on disparate chip materials and made with disparate technologies, such as Si, SiC, SiN, InP, GaAs, Liquid Crystal, etc. This integrated system can be stacked in as shown in FIG. 4, with the connections between circuit elements being formed using BGA/C4/micro-BGA, through substrate (or silicon) vias (TSVs), and micro-TSVs. Physical connections between layers can be through solder or oxide bonding techniques. In one example, the control circuitry 240 comprises a printed circuit board and the imaging systems 130 and projection systems 135 may comprise chips that are physically and electronically connected to the printed circuit board

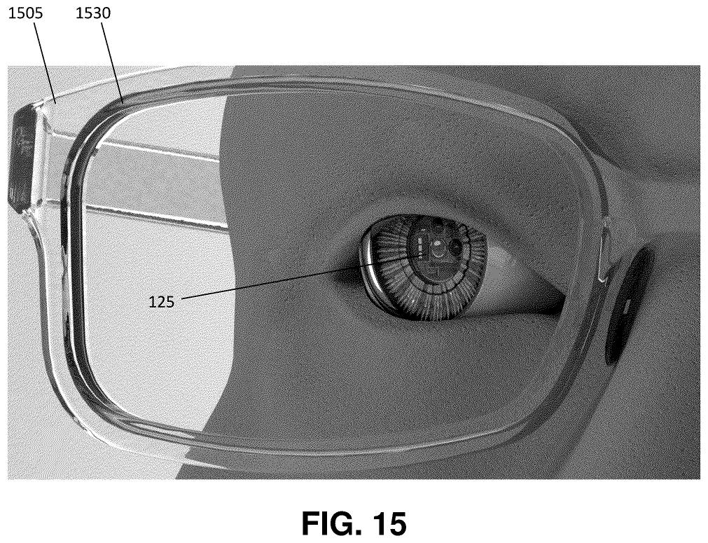

In embodiments, the wireless charging coil 220 comprises one or more inductive coupling coils and the power source 235 comprises a rechargeable battery that can be wirelessly recharged through inductive coupling using the wireless charging coil 220. In this regard, although not shown, the wireless charging coil 220 may be operatively connected to the power source 235, e.g., directly or indirectly via the control circuitry 240. The wireless charging coil 220 may be used to wirelessly charge the power source 235 using an external device, e.g., as shown in FIG. 15. The wireless communication antenna 225 may be in the haptics 215, in the central body 205, or in a chip or chip stack that contains the control circuitry 240, or any combination thereof. In some embodiments, the power source 235 does not store an appreciable amount of electrical power or the power source 235 is omitted altogether. In such embodiments, the ocular device 125 may be configured to use the electronics only when being wirelessly powered by an external device such as eyeglasses 1505 show in FIG. 15. In such embodiments, the ocular device 125 may be configured to automatically turn on or turn off the electronics, respectively, based on the user donning or doffing the external device that wirelessly provides power to the ocular device 125.

In various embodiments, the ocular device 125 is specifically configured via its shape and size to be implanted in a capsular bag of an eye, a ciliary sulcus of an eye, or an anterior chamber of an eye, e.g., as shown and described in US Patent Application Publications No. 2024/0138673 and 2025/0099299, the contents of each of which are incorporated by reference herein in their entirety. In this regard, in the ocular device 125 sub-circuit chips of the electronics 212 may be thinned using wafer thinning techniques to be thin enough such that the entire system is such that the thickness dimension TH satisfies the expression 1 mm<=TH<=3 mm. These techniques are employed in stacked memory chips with wafers thinned to less than 20 μm thick and bonded to other wafers and connecting micro-TSVs are made between active layers that are 10 μm to 20 μm tall. The ocular device 125 may be constructed such that the width dimension W satisfies the expression 1 mm<=W<=10 mm. An ocular device 125 having these dimensions TH and W is suitable for implanting an eye (e.g., a human eye), such as shown in FIG. 5 and as shown and described in US Patent Application Publications No. 2024/0138673 and 2025/0099299.

In accordance with aspects of the present disclosure, the images that are projected onto the retina by the respective projection systems 135 correspond to the incoming light received at the respective imaging systems 130, such that the ocular device 125 may be used as an artificial vision system that can be used to treat vision disorders such as blindness. By using at least two imaging systems 130 and projection systems 135, e.g., one imaging system and projection system pair for frontal vision and one imaging system and projection system pair for peripheral vision, embodiments may be used to provide near perfect vision to the user.

An example of an ocular device including two imaging systems 130 and two projection systems 135 has been described thus far with respect to FIGS. 1-5. Implementations of the ocular device according to the present disclosure are not limited to two imaging systems 130 and two projection systems 135, and other numbers of imaging systems 130 and/or projection systems 135 may be used.

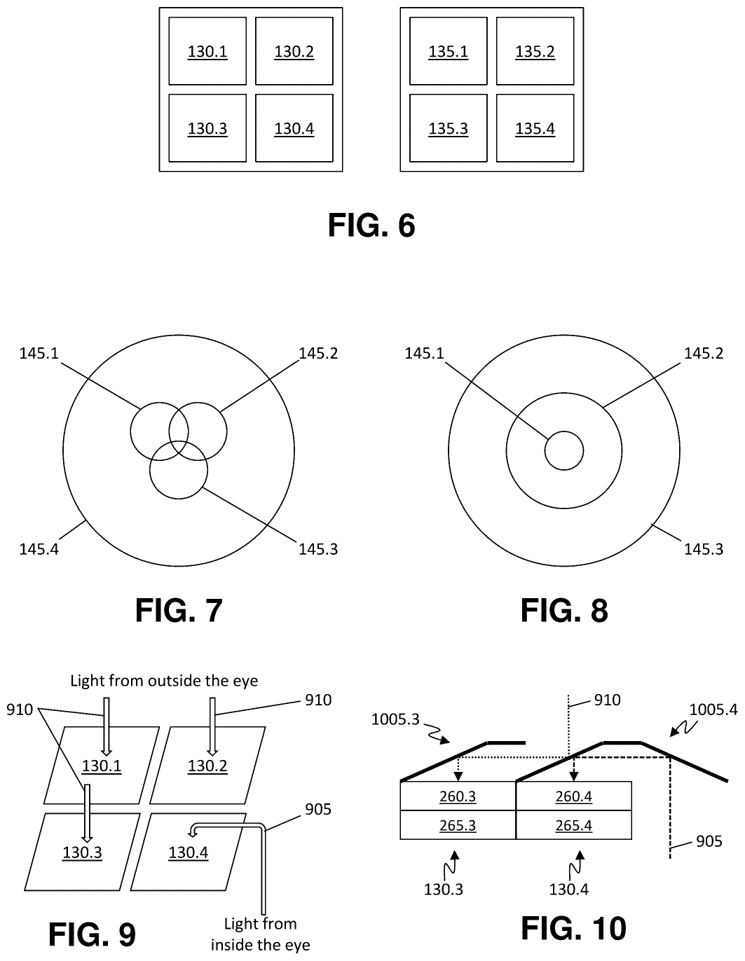

FIG. 6 diagrammatically shows an exemplary implementation of an ocular device in accordance with aspects of the present disclosure. As shown in FIG. 6, the ocular device 125 may include four imaging systems 130.1, 130.2, 130.3, and 130.4 and four projection systems 135.1, 135.2, 135.3, and 135.4.

FIG. 7 diagrammatically shows an example of projection regions on the retina using the ocular device of FIG. 6. In the example shown in FIG. 7, the first projection system 135.1 projects light (images) onto a first region 145.1 of the retina based on light received at the first imaging system 130.1, the second projection system 135.2 projects light (images) onto a second region 145.2 of the retina based on light received at the second imaging system 130.2, the third projection system 135.3 projects light (images) onto a third region 145.3 of the retina based on light received at the third imaging system 130.3, and the fourth projection system 135.4 projects light (images) onto a fourth region 145.4 of the retina based on light received at the fourth imaging system 130.4. In this example, the first, second, and third projection systems 135.1-135.3 combine their projected images to form the frontal vision, and the fourth projection system 135.4 projects images for the peripheral vision. For example, the first, second, and third projection systems 135.1-135.3 may project relatively high-resolution images onto cone regions of the retina, and the fourth projection system 135.4 may project relatively low-resolution images onto the rod regions of the retina. As with other examples described herein, the respective projection systems 135.1-135.4 may be controlled so that the images do not overlap even though the regions 145.1-145.4 might overlap. This may be accomplished, for example, by using data that maps regions of the retina, optical characteristics of respective lenses, and selectively controlling which portions (e.g., pixels) of the respective light generating devices are used for emitting light.

FIG. 8 diagrammatically shows another example of projection regions on the retina using the ocular device of FIG. 6. In the example shown in FIG. 8, the first projection system 135.1 projects light (images) onto a first region 145.1 of the retina based on light received at the first imaging system 130.1, the second projection system 135.2 projects light (images) onto a second region 145.2 of the retina based on light received at the second imaging system 130.2, the third projection system 135.3 projects light (images) onto a third region 145.3 of the retina based on light received at the third imaging system 130.3, and the fourth projection system 135.4 does not project light onto the retina. In this example, the first region 145.1 is defined within a first range of off-axis angles (e.g., off-axis angles within a range of +/−2 degrees) and the first projection system 135.1 projects at a first resolution (e.g., 30 pixels per degree) in this first region. In this example, the second region 145.2 is defined within a second range of off-axis angles (e.g., off-axis angles within a range of +/−15 degrees) and the second projection system 135.2 projects at a second resolution (e.g., 12 pixels per degree) in this second region. In this example, the third region 145.3 is defined within a third range of off-axis angles (e.g., off-axis angles within a range of +/−60 degrees) and the third projection system 135.3 projects at a third resolution (e.g., 5 pixels per degree) in this third region. As with other examples described herein, the respective projection systems 135.1-135.3 may be controlled so that the images do not overlap even though the regions 145.1-145.3 might overlap. This may be accomplished, for example, by using data that maps regions of the retina, optical characteristics of respective lenses, and selectively controlling which portions (e.g., pixels) of the respective light generating devices are used for emitting light. For example, although the second region 145.2 is defined within a second range of off-axis angles (e.g., off-axis angles within a range of +/−15 degrees), the second projection system 135.2 may be configured to project only into portions of the second region 145.2 that do not overlap the first region 145.1. Similarly, although the third region 145.3 is defined within a third range of off-axis angles (e.g., off-axis angles within a range of +/−60 degrees), the third projection system 135.3 may be configured to project only into portions of the third region 145.3 that do not overlap the first region 145.1 and/or the second region 145.2.

In the example shown in FIG. 8, different ones of the imaging systems 130.1-130.3 may include different lenses with different optical characteristics, e.g., to define different fields of view outside the eye in which the ocular device is implanted. Moreover, different ones of the projection systems 135.1-135.3 may include different lenses with different optical characteristics, e.g., to define different fields of view (e.g., projection areas) inside the eye.

FIG. 9 diagrammatically shows an exemplary arrangement of the imaging systems of ocular device of FIGS. 6 and 8 in accordance with aspects of the present disclosure. As described in the example associated with FIG. 8, the ocular device projects images onto the retina using three of the four projection systems 135.1-135.3 and does not project images onto the retinac using the fourth projection system 135.4. In this type of implementation, the ocular device may be configured to receive light from outside the eye using the three imaging systems 130.1-130.3 that are respectively associated with the three projection systems 135.1-135.3, and may be configured to use the fourth imaging system 130.4 for another purpose. In one example, the ocular device is configured to use the fourth imaging system 130.4 to receive light from inside the eye, as indicated in FIG. 9 by arrow 905 that represents light from inside the eye in which the ocular device is implanted and arrows 910 that represent light from outside the eye in which the ocular device is implanted.

FIG. 10 diagrammatically shows an exemplary arrangement of the third imaging system 130.3 and the fourth imaging system 130.4 from FIG. 9 in accordance with aspects of the present disclosure. In this example, the third imaging system 130.3 includes a lens 260.3 and image sensor 265.3, and the fourth imaging system 130.4 includes a lens 260.4 and image sensor 265.4. In this example, the third imaging system 130.3 includes structure 1005.3 that causes light from outside the eye (represented by arrow 910) to impinge upon the lens 260.3 and image sensor 265.3, and the fourth imaging system 130.4 includes structure 1005.4 that causes light from inside the eye (represented by arrow 905) to impinge upon the lens 260.4 and image sensor 265.4. The respective structures 1005.3 and 1005.4 may comprise one or more reflective surfaces and/or prisms that define a circuitous optical path, e.g., as described in U.S. application Ser. No. 19/195,971. In this manner, one or more of the imaging systems may be used to collect light from inside the eye in which the ocular device is implanted, which may be used in mapping healthy and unhealthy (e.g., damaged) portions of the retina of the eye in which the ocular device is implanted, which may be used to determine regions onto which to project images as described herein.

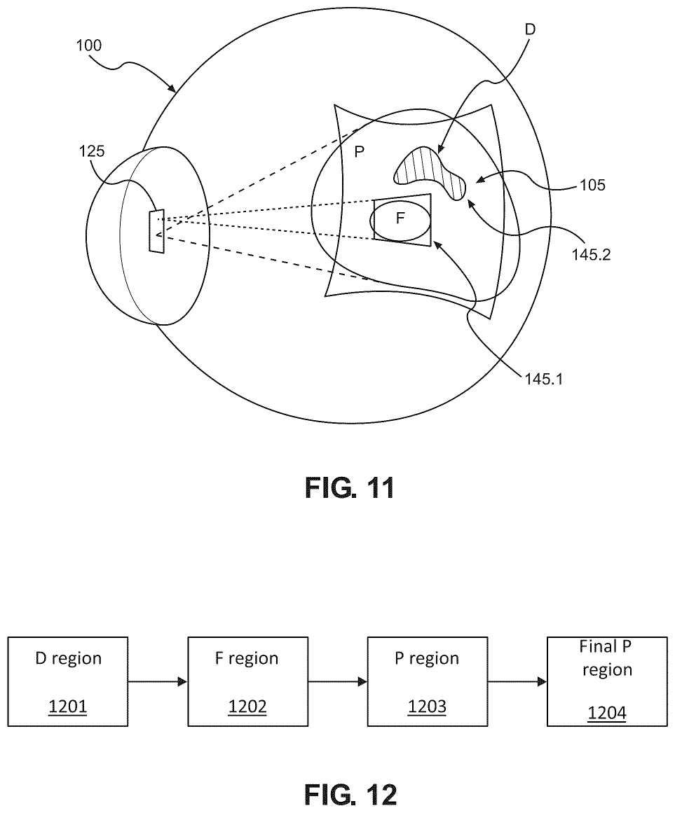

FIG. 11 diagrammatically shows an example of an ocular device accommodating a damaged area of the retina in accordance with aspects of the present disclosure. In this example, the ocular device 125 is implanted in the anterior region of the eye 100 and is configured to project images onto the first region 145.1 of the retina 105 corresponding with frontal vision (indicated by “F”) and to project images on the second region 145.2 of the retina corresponding with peripheral vison (indicated by “P”). In this example, the retina 105 includes a damaged region indicated by “D”. In this example, the retina tissue in the damaged region is incapable of being used for vision and thus represents a blind spot in the person's vision. In this example, the ocular device 125 reduces its power consumption by not projecting light onto the damaged region since doing so would have no effect on the vision of the user.

In embodiments, and as described herein, the ocular device 125 stores data that is based on a mapping of the retina 105 for a particular eye of a particular user and that is used to control the operation of the first projection system 135.1 and the second projection system 135.2. the mapping of the retina upon which this data is based may include a definition of the first region 145.1 (“F”), the second region 145.2 (“P”), and the damaged region (“D”). Using such data, the ocular device 125 may be configured to not project light onto a damaged region. One method of doing this is to selectively turn off (e.g., not use) individual light emitting elements (e.g., LED pixels) in one or more of the projection systems that are mapped to the damaged region. By selectively not using individual light emitting elements in this manner, the power consumption of the ocular device 125 is reduced compared to when all the light emitting elements are used to project. The mapping may be determined in the manner described in US Patent Application Publication No. 2025/0099299. In another example, the mapping may additionally or alternatively be determined by capturing images of the retina in which the ocular device is implanted, e.g., using an arrangement similar to that shown in FIGS. 9 and 10 in which at least one of the imaging systems is configured to receive light from inside the eye. In this example, images of the retina captured by the ocular device 125 may be analyzed using image analysis techniques to differentiate between healthy regions and damaged regions of the retina. The image analysis may be performed by the control circuitry 240 of the ocular device 125, or may be performed by an external computing device (e.g., smartphone, tablet computer, laptop computer, desktop computer, server, etc.) that communicates with the ocular device 125, e.g., via the wireless communication antenna 225.

FIG. 12 shows a flowchart of an exemplary method in accordance with aspects of the present disclosure. The method illustrates an example of an algorithm used in determining how many pixels to display on the projection system responsible for the peripheral vision, where D is the damaged area of the retina, F is the central vision projection, and P is the peripheral vision projection as shown in FIG. 11. Steps of the method (also referred to as operations) may be carried out using the ocular device 125.

In this example, and as shown in the FIG. 11, the damaged region D is wholly within the second region 145.2 corresponding to peripheral vision. Accordingly, at step 1201 the control circuitry 240 of the ocular device 125 determines which pixels in the second projection system 135.2 are mapped to the area of the retina corresponding to the damaged region D. At step 1202 the control circuitry 240 of the ocular device 125 determines which pixels in the second projection system 135.2 are mapped to the first region 145.1 correspond to frontal vision F. At step 1203 the control circuitry 240 of the ocular device 125 determines which pixels in the second projection system 135.2 are mapped to the second region 145.2 correspond to peripheral vision P. At step 1204 the control circuitry determines which pixels in the second projection system 135.2 to utilize in project an image by subtracting the pixels identified at steps 1201 and 1202 from the pixels identified at step 1203. In this manner, the ocular device 125 uses the second projection system 135.2 to project only onto areas in the second region 145.2 that do not overlap the first region 145.1 and that do not overlap the damaged region D.

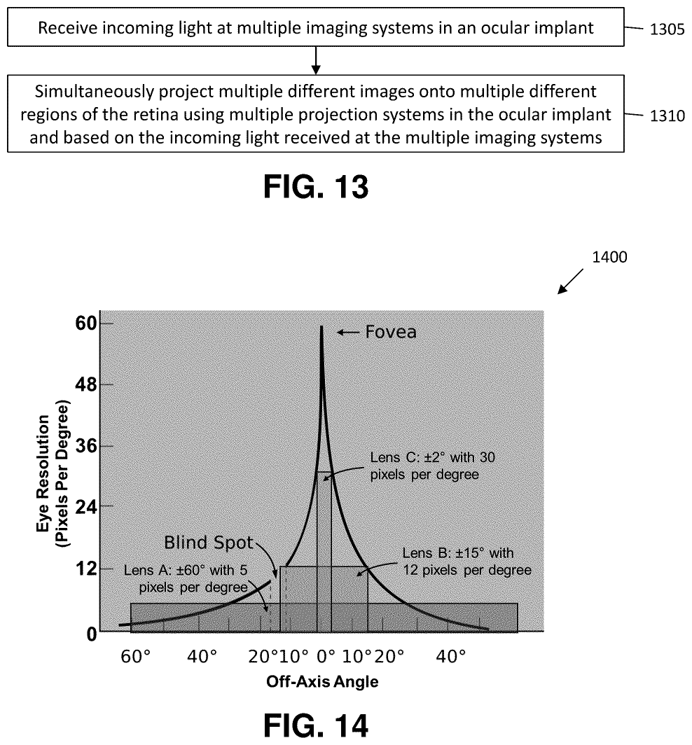

FIG. 13 shows a flowchart of an exemplary method in accordance with aspects of the present disclosure. Steps of the method (also referred to as operations) may be carried out using the ocular device 125. Step 1305 includes receiving incoming light at multiple imaging systems 130 in an ocular implant 125. Step 1310 includes simultaneously projecting multiple different images onto multiple different regions of the retina using multiple projection systems 135 in the ocular implant 125 and based on the incoming light received at the multiple imaging systems 130.

FIG. 15 illustrates wireless charging in accordance with aspects of the present disclosure. In various embodiments, an external device comprising eyeglasses 1505 includes a wireless charging system 1530 that is configured to wirelessly charge the power source 235 of the ocular device 125. In one example, the wireless charging system 1530 comprises a charging coil that cooperates with the charging coil 220 in the ocular device 125 to perform inductive charging of the power source 235 of the ocular device 125. The eyeglasses 1505 may include its own power source, such as an on-board battery or wired connection to an external power source, that powers the wireless charging of the power source 235 of the ocular device 125. In embodiments in which the power source 235 does not store an appreciable amount of power or is omitted, the wireless charging system 1530 is configured to wirelessly provide power to the ocular device 125 for operating the electronics during the time that the power is wirelessly provided.

In some embodiments, the ocular device 125 comprises a contact lens that is configured to be placed on an exterior of the eye or an implantable contact lens that is configured to be implanted in the eye. In these embodiments, the electronic imaging systems, the electronic projection systems, and the other electronics are packaged in a contact lens shaped form factor. In these embodiments, the electronic imaging systems, the electronic projection systems, and the other electronics function in a similar manner as described herein. These components may have different sizes, shapes, and locations in a contact lens compared to an the device shown in FIG. 4, for example, due to the different form factors of contact lenses.

Various embodiments include methods of using an artificial vision system in accordance with aspects of the present disclosure. One such method is described in FIG. 13. Various embodiments include methods of implanting an artificial vision system in accordance with aspects of the present disclosure. Such methods may include implanting the ocular device into the eye of the user.

Some embodiments of the ocular device 125 according to aspects of the present disclosure use a single shared image sensor for the multiple imaging systems 130 (e.g., multiple different lenses arranged over different sections of a single CMOS image sensor) and a shared light projecting device for the multiple projection systems 135 (e.g., multiple different lenses arranged over different sections of a single LED panel). First, a system that uses a single imaging chip with multiple imaging systems 130 only needs to power the regions of the imaging chip that are involved with the display/reception of the image data. Second, as described above, when the system is designed to make use of the eyes natural resolution profile, there are great savings in pixels required.

Depending on the optics system chosen, the images may differ with respect to the magnification, area of interest, field of view, monochrome or polychrome, depth of field, etc. The same is true for a similarly sized LED/projection displays.

Furthermore, in such an arrangement, power consumption can be further reduced by turning off pixels that are not being used. For example, a device using multiple imaging systems 130 on a single CMOS image sensor can take high-resolution images from a narrow field of view optical system (for central vision), lower-resolution images from a wide field of view optical system (for peripheral vision), and images of the retina using an optical system pointed in the reverse direction (e.g., similar to that shown at imaging system 130.4 in FIG. 9) that can be used to monitor the health of the retina or the progression of the disease over time.

In conjunction, multiple optical systems on a single display chip can be used to project images onto undamaged regions of the retina (D), e.g., central vision (F) and peripheral vision (P). To avoid overlapping images or placing the image on the damaged region, an algorithm can be used to both optimize placement of the images and selectively turn on/off pixels as described in FIG. 12. Finally, one or more optical lens system can be used to monitor the health of the retina by collecting light from behind the implant, e.g., as shown in FIG. 9. A rear-facing projector (LED or other) can provide the light source for the retinal imaging (like a flashbulb on a camera). Depending on the imaging optical lens system, this can take a wide-angle or a narrow field-of-view image as needed. This can also be two wide-field images digitally stitched together. By incorporating this image collection into the startup or shutdown routine of the device, a series of these images would illustrate the progression of a retinal disease or the monitoring of retinal health. Finally, this image collection, in addition to the patient's input, would provide valuable information in calibrating a rear-facing projector displaying the output from a front-facing camera as a treatment for de facto blindness from late-stage advanced macular degeneration and other retinal diseases that damage the retina.

In another example in accordance with aspects of the present disclosure, the ocular device 125 includes a single CMOS image sensor with 400×400 pixels shared by four distinct imaging systems 130 referred to in this example as optical systems for imaging (OSI 1-4) that collect light on the 200×200 photoreceptors on their respective quadrants of the CMOS image sensor. Such an embodiment may resemble FIG. 6, for example, except that the four imaging systems 130.1-130.4 share a single CMOS image sensor instead of each having its own dedicated CMOS image sensor. Each optical system for imaging (OSI) has its own lens(es) and mirror(s) or other reflective/focusing elements, and OSI 1 and OSI 2 collect light from outside of the eye, while OSI 3 and OSI 4 collect light from the back of the eye (retina), e.g., similar to FIG. 9 in which some of the imaging systems collect light from outside the eye and some of the imaging systems collect light from inside the eye. In this example, OSI 1 has a wide field of view and collects the full field of view that a patient would have with their single-eye peripheral vision. This image is collected on 200×200 pixels. In this example, OSI 2 has a narrow field of view and collects the equivalent to the high-resolution central vision. This image is also collected on 200×200 pixels. In this example, OSI 3 and OSI 4 do the same, except they collect images of the retina and are used to evaluate the health of the retina. Since OSI 1-4 are passive optical elements, they all operate simultaneously and are always on, and the image sensor collects all 4 images simultaneously. The control circuitry 240 of the ocular device 125 in this example may choose to discard information from any of the pixels, and the expectation is that the pixels from OSI 1-2 are always in use, while the pixels from OSI 3-4 are often discarded except during specific diagnostic routines, where light shined in the eye, either from the onboard LED chip or from an external light source, can illuminate the retina.

In another example in accordance with aspects of the present disclosure, the ocular device 125 includes single LED chip with 400×400 pixels shared by four distinct projection systems 135 referred to in this example as optical systems for projection (OSP 1-4) that project light from the 200×200 pixel on their respective quadrants of the LED chip. Such an embodiment may resemble FIG. 6, for example, except that the four projection systems 135.1-135.4 share a single LED chip instead of each having its own dedicated LED chip. Each optical system for projection (OSP) has its own lens(es) and mirror(s) or other reflective/focusing elements. In this example, quadrants 1 and 3 (Q1 and Q3) project the central and peripheral vision, respectively and quadrants 2 and 4 (Q2 and Q4) are unused to save power. In this example, OSP 1 has a narrow field of view and projects onto an arbitrary portion of the retina by using a subset of the 200×200 pixels available in the quadrant. In this example, OSP 3 has a wide field of view and projects the full field of view onto the patient's retina using the full set of 200×200 pixels, except where the central vision would be projected and except for the damaged area of the retina. Note in this and the other examples, the primary power savings is not in which areas of the chips can be turned off, that is additional savings, the primary power savings is using specialized lens that support the appropriate pixels/degree and the appropriate field of vision such that the combined operations of the lens matches the natural resolution profile of the eye. In addition, as just described, for single-chip embodiments, multiple imaging/projecting lenses can be employed even to do things such as in situ retinal imaging.

In various embodiments, the CMOS image sensor and LED projector systems work in tandem based on pre-defined modes defined by an FPGA or ASIC controller. Many pre-defined modes are possible, and one pre-defined mode is described above, where with a single CMOS image sensor with 400×400 pixels that has four distinct optical systems (OSI 1-4) that collect light on the 200×200 photoreceptors on their respective quadrants of the chip. Each optical system (OSI) has its own lens(es) and mirror(s) or other reflective/focusing elements, and OSI 1 and 2 collect light from outside of the eye, while OSI 3 and OSI4 collect light from the back of the eye (retina).

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. An electronic device configured to be implanted in or on an eye, the electronic device comprising:
multiple imaging systems configured to receive incoming light at the eye; and
multiple projection systems configured to simultaneously project multiple different images onto multiple different regions of a retina of the eye based on the incoming light received by the imaging systems.

2. The electronic device of claim 1, wherein the different regions of the retina include:
a first region associated with frontal vision; and
a second region associated with peripheral vision.

3. The electronic device of claim 2, wherein the projection systems comprise:
a first projection system configured to project first images onto the first region at a first resolution; and
a second projection system configured to project second images onto the second region at a second resolution that is lower than the first resolution.

4. The electronic device of claim 1, wherein respective ones of the different regions are defined based on respective ranges of off-axis angles in the eye.

5. The electronic device of claim 1, wherein the imaging systems share a single imaging sensor and each respective one of the imaging systems has a respective imaging lens over a respective portion of the single imaging sensor.

6. The electronic device of claim 1, wherein the projection systems share a single light projection device and each respective one of the projection systems has a respective projection lens over a respective portion of the single light generation device.

7. The electronic device of claim 1, wherein each respective one of the imaging systems has a respective imaging lens and a respective imaging sensor.

8. The electronic device of claim 7, wherein different ones of the respective imaging sensors have different numbers of pixels.

9. The electronic device of claim 1, wherein each respective one of the projection systems has a respective projection lens and a respective light generation device.

10. The electronic device of claim 9, wherein different ones of the respective light generation devices have different numbers of pixels.

11. The electronic device of claim 1, wherein the electronic device is configured to be implanted in a capsular bag of the eye, a ciliary sulcus of the eye, or an anterior chamber of the eye.

12. The electronic device of claim 1, wherein the electronic device comprises a central body and haptics extending from the central body.

13. The electronic device of claim 12, wherein the imaging systems and the projection systems are contained in a chip or chip stack, or attached to a printed circuit board, in the central body.

14. The electronic device of claim 13, further comprising control circuitry, a power source, a wireless communication system, and a wireless charging system.

15. The electronic device of claim 1, wherein:
at least two of the imaging systems are configured to receive light coming from outside the eye; and
at least one of the imaging systems is configured to receive light coming from inside the eye.

16. A method comprising determining a damaged area of the retina by analyzing images defined by the light received by the at least one of the imaging systems in the electronic device of claim 15.

17. A method of using the electronic device of claim 3, the method comprising:
projecting the first images using a first a light generation device in the first projection system; and
projecting the second images using a second a light generation device in the second projection system, wherein the projecting the second images comprises not using pixels in the second light generation device that correspond to the first region.

18. The method of claim 17, wherein the projecting the second images comprises not using other pixels in the second light generation device that correspond to a damaged region of the retina.

19. The electronic device of claim 1, wherein the multiple different regions of the retina include:
a first region associated with frontal vision;
a second region associated with peripheral vision; and
a middle region between the first region and the second region.

20. The electronic device of claim 19, wherein the multiple projection systems comprise:
a first projection system configured to project images onto the first region at a first resolution;
a second projection system configured to project images onto the second region at a second resolution that is less than the first resolution; and
a third projection system configured to project images onto the middle region at a third resolution that is less than the first resolution and greater than the second resolution.

21. The electronic device of claim 1, wherein the electronic device comprises one of: a contact lens that is configured to be placed on an exterior of the eye; or an implantable contact lens that is configured to be implanted in the eye.
